(19) United States
(12) Patent Application Publication
(10) Pub. No.: US 2023/0225649 A1
(43) Pub. Date: Jul. 20, 2023

(54) GRAPH MODEL-BASED BRAIN FUNCTIONAL ALIGNMENT METHOD

(71) Applicant: ZHEJIANG LAB, Hangzhou (CN)

(72) Inventors: Yu ZHANG, Hangzhou (CN); Chaoliang SUN, Hangzhou (CN); Zhichao WANG, Hangzhou (CN); Haotian QIAN, Hangzhou (CN); Jun LI, Hangzhou (CN); Jingsong LI, Hangzhou (CN)

(21) Appl. No.: 18/125,645

(22) Filed: Mar. 23, 2023

Related U.S. Application Data
(63) Continuation of application No. PCT/CN2022/113906, filed on Aug. 22, 2022.

(30) Foreign Application Priority Data
Sep. 17, 2021 (CN) 202111090208.3

(51) Int. Cl. A61B 5/16; A61B 5/00

(52) U.S. Cl. CPC A61B 5/16; A61B 5/7264

(57) ABSTRACT

Disclosed is a graph model-based brain functional alignment method. The method includes: mapping high-dimensional functional brain imaging data to a two-dimensional time-series matrix by taking brain functional activity signals of a subject under a specific cognitive function state as input , constructing a model based on graph convolutional networks to distinguish different cognitive function states, generating a brain activation distribution priori graph by a meta analysis method to assist in predicting a specific brain function activation mode of each subject, combining the two to map functional brain imaging data of each subject to a shared representation space applicable to a large-scale group, and finally achieving accurate brain function alignment between subjects. According to the method, graph representation information generated in the shared representation space can also be used for accurately predicting the brain function state and behavioral index of the subjects.

### CROSS-REFERENCE TO RELATED APPLICATIONS

The present disclosure claims priority to Chinese Patent Application No. 202111090208.3, submitted to the China National Intellectual Property Administration on Sep. 17, 2021 and entitled “GRAPH MODEL-BASED BRAIN FUNCTIONAL ALIGNMENT METHOD”, which is incorporated herein by reference in its entirety.

### TECHNICAL FIELD

The present disclosure relates to the fields of medical imaging and deep learning, and in particular to a method for automatically registering a functional brain image in a task state and aligning functionality between subjects.

### BACKGROUND

In the research of functional magnetic resonance imaging (fMRI), functional brain imaging data of a plurality of subjects are used to perform group analysis, which accounts for a more and more proportion. On one hand, group analysis based on a plurality of subjects can effectively verify the universality and effectiveness of the research result on different subjects, and also can increase the effect dose of statistical analysis in the functional brain image analysis. On the other hand, due to different anatomical structures of different subjects and different functional region positioning, it is necessary to register functional brain imaging data of different subjects. For example, all the subjects are registered on an image template in a common standard space, the functional brain images of different subjects are further analyzed and compared, and a statistical analysis model is created, so that the statistical analysis result of the brain function activation mode in a specific cognitive function state is obtained.

An existing functional brain image registration method mainly includes two categories: registration based on functional image formation (such as EPInorm) and registration based on structural morphology (such as T1norm). According to the registration method based on functional image information, such as registration of an echo planar imaging sequence (EPInorm), the functional image of the subject is registered on a functional image template (that is, EPI template) in the standard space directly through linear or non-linear transformation. However, the EPI image will be geometrically distorted due to the problem of non-uniformity of the magnetic field, and it is necessary to perform geometric correction to ensure the correspondence with the grain anatomical structure. In additional, functional brain imaging data often has the problems of low spatial resolution ratio and low organization contrast ratio, lacks obvious brain anatomical structure detailed information, and is prone to cause excessive correction in the registration process. For example, a signal in an irrelevant brain region, even a white matter region is used to fill the signal of the missed brain functional region, resulting in that the final registration accuracy is not high.

The registration method based on structural morphology indirectly completes registration of functional brain images between subjects by structural magnetic resonance imaging (sMRI) (T1w), and includes the general steps as follows: 1) registering functional brain imaging data of a subject to a space where the current brain structure image of the subject is located through rigid body transformation or affine transformation; 2) registering the brain structure image of the subject to a brain structure image template of a standard space through non-linear transformation, and saving a registration parameter from an image space of each subject to the standard space; and 3) applying the registration parameter obtained in the step 2) to functional brain imaging data of the subject, and finally realizing registration of the functional brain imaging data of all the subjects from a subject space to the standard space. The method performs registration by using sMRI with high resolution ratio, so that the registration accuracy higher than that of EPInorm can be achieved. However, due to the problems of geometric distortion of the functional brain image and the difference of gray matter organization contrast, the cross-modal registration from the functional brain image to the structural brain image faces great challenges. In addition, certain differences may be present in the anatomical position, size and shape of the brain functional regions of different subjects, resulting in imperfect correspondence between the brain anatomical structure and the brain functional region, so that the registered brain functional regions are not completely consistent on all the subjects. The difference between the brain anatomical structures and the brain functional regions has been verified in numerous researches. Therefore, according to the registered functional brain imaging data acquired by the registration method based on structural morphology, although the accurate correspondence between the subjects is realized in the brain anatomical structure, deviation still may occur in the brain functional representation (such as the brain functional region and the brain activation mode corresponding to a specific cognitive functional state), and functional alignment between the subjects in a strict sense cannot be realized, so that the effect dose of statistical analysis is affected, especially for the higher cognitive functions such as language and working memory showing great subject difference, the functional alignment of the brain image data is often more important than anatomical alignment.

The goal of brain functional registration is that in order to better cope with the brain functional difference between subjects, complete correspondence in the brain anatomical position or structural morphology information of all the subjects is required, and one-to-one correspondence of the brain functional activities (such as representative brain functional region and brain functional activation graph) of all the subjects in the same cognitive functional state is also required. The advantages of the brain functional registration are: the effect dose of the statistical analysis during group analysis the detection sensitivity of the brain activation region can be enhanced, and the brain functional registration can be used to accurately predict the brain functional activity state and the behavioral index of each subject.

Chen et al., “A reduced-dimension fMRI shared response model,” in Advances in Neural Information Processing Systems 28 (eds. Cortes, C., Lawrence, N. D., Lee, D. D., Sugiyama, M. & Garnett, R.) 460-468,2015.

Vince D. Calhoun et al., “The impact of T1 versus EPI spatial normalization templates for fMRI data analyses,” Hum. Brain. Mapp. 38,5331-5342,2017.

H. Xu et al., “Regularized hyperalignment of multi-set fMRI data,” in Proc. Statistical Signal Processing Workshop, pages 229-232. IEEE, 2012.

J. D. G. Watson et al., “Area V5 of the human brain: evidence from a combined study using positron emission tomography and magnetic resonance imaging,” Cereb. Cortex, 3:79-94, 1993.

### SUMMARY

For the above problems in existing methods, the present disclosure provides a graph model-based brain functional alignment method. On the basis of completing the brain structural morphology registration, functional brain imaging data of all subjects in the same cognitive function state are mapped to the same representation space by using an artificial intelligent algorithm and supervised learning and under the guidance of distinguishing brain functional activity signals in different cognitive functional states, thereby ensuring that brain functional activation modes between different subjects have a good correspondence.

The present disclosure adopts the following technical solution: a graph model-based brain functional alignment method includes: mapping high-dimensional functional brain imaging data to a two-dimensional time-series matrix by taking brain functional activity signals of a subject in a specific cognitive function state as an input and taking a brain graph model as a basis, constructing a model based on graph convolutional networks to distinguish different cognitive function states, and obtaining a brain activation distribution priori graph by a meta analysis method to predict a brain function activation mode of each subject so as to map functional brain imaging data of each subject to a shared representation space applicable to a large-scale group, and finally achieving accurate brain function alignment between subjects. The method includes the following steps.

(1) according to the design of a cognitive experimental paradigm, recording a cognitive function state on each time frame in a functional brain imaging data set;

(2) registering functional brain imaging data of all subjects to an image template of a common standard space based on structural morphology information, and ensuring the correspondence of the subjects on a brain anatomical structure;

(3) creating a unified brain graph model under the standard space using brain atlas and brain connectomes;

(4) converting an original feature of high-dimensional brain function images into a two-dimensional time-series matrix by using the brain graph model in the step (3), wherein the first dimension represents different brain regions, and the second dimension represents different time frames; adding the time-series matrix as a graph signal into the brain graph model for representing brain functional activity signals on each brain region;

(5) calculating a graph Laplacian matrix of the brain graph model, obtaining eigenvalues and eigenvectors of the graph Laplacian matrix using spectral decomposition, transforming the graph signal in the step (4) from an original image space to a spectral domain defined by the brain graph model through graph Fourier transformation, applying graph convolution operations and spectral analysis on the brain functional activity signals, and learning graph representations of brain activity by creating a model based on graph convolutional networks;

(6) for the researched cognitive experimental paradigm, using a meta analysis method to obtain priori knowledge of a brain function activation mode of a cognitive function from the published related research, and generating a brain activation distribution priori graph;

(7) adding a term in the loss function for representing the correspondence degree of the brain activation distribution priori graph in each brain region in a target function of the model based on graph convolutional networks by combining the prior knowledge in the step (6);

(8) training the model based on graph convolutional networks, extracting feature information of the last convolutional layer as graph representation information of the brain functional activity signal, where the graph representation information maps functional brain imaging data of different subjects to the same representation space to realize brain function alignment of the subjects and generate a brain function activation graph of the subjects; and

(9) displaying the brain function activation graph of the subjects on a displayer.

Further, in the step (2), the specific operation of registration based on the structural morphology information is: registering the functional brain imaging data of each subject to a space where a brain structure image of the subject is located through rigid body transformation or affine transformation to realize cross-modal registration in the same subject; registering the structure image of the subject to a structure image template of a standard space by non-linear transformation, and saving a registration parameter of each subject to the standard space; and applying the registration parameter to the functional brain image of the subject to realize registration from an subject space to the standard space.

Further, the specific construction process of the brain graph model is: parcellating the whole cerebral cortex and subcutaneous substructure into spatially separated brain regions by using a well-established brain atlas; calculating the connectivity pattern between different brain regions using diffusion magnetic resonance imaging (dMRI) or fMRI; and constructing a brain graph model, where a node set V is formed by brain regions extracted by the brain atlas, and an edge set E is defined by brain connectome obtained through calculation.

Further, the brain atlas includes an anatomical, function and multi-modal brain atlas; and the brain connectome comprises an anatomical connectivity derived from diffusion tractography using diffusion MRI, a functional connectivity based on resting-state functional MRI, and a structural connectivity based on structural covariance using structure MRI and morphological feature covariance.

Further, the graph signal is calculated as follows: calculating the mean value and variance, averaged time series and principal components of the brain functional activity signals in each brain region in the corresponding cognitive function states.

Further, the spectral analysis of the brain functional activity signals is specifically as follows: calculating the graph Laplacian matrix of the brain graph model L=I−D^{-1/2}AD^{-1/2}, where I represents a unit matrix, A represents a connection relationship between nodes, and D represents the connectivity on each node; obtaining the eigenvalues {λᵢ} and eigenvectors {vᵢ} by using spectral decomposition of the graph Laplacian matrix Lv=λv, and performing graph Fourier transformation \tilde{x}=U^{T}x on the basis U=(v₁, v₂, . . . vₙ), where the eigenvalue {λᵢ} represents different frequency bands of graph Fourier transformation, the eigenvector {vᵢ} represents the transformation under the corresponding frequency band, n represents the total number of brain regions, represents the original graph signal, \tilde{x} represents the transformed signal, and T represents the transpose operation; and transforming the graph signal from an image space domain to a spectral domain defined by the brain graph model through graph Fourier transformation, and applying graph convolution operations in the spectral domain x*_{G}g_{θ}=Ug_{θ}U^{T}x, where g_{θ} represents a convolution kernel, and *_{G} represents the convolutional operation defined on the brain graph model, and constructing a model based on graph convolutional network by taking the graph convolution operations.

Further, the calculation of the group priors of the brain activation comprises: using meta analysis tools to extract the peak coordinates of significantly activated brain regions under the cognitive task or using the same experimental paradigm from an existing database, generating a Gaussian smoothed brain activation map on each peak, and generating a prior distribution of brain activation by using a statistical analysis.

Further, a target function of the model based on graph convolutional networks comprises two items: a target function of the model based on graph convolutional networks comprises two items: the first item is a cross entropy loss for predicting the cognitive state at each time frame, and the second item is a masked mean square error loss function for constraining graph representation information to fit priori knowledge activated by a brain function on a key brain region as much as possible. The final target loss function Loss formed by the two items is specifically as follows:

$$Loss = {\sum }_{i , k} {y}_{ik} log ( {p}_{ik} ) + α {\sum }_{i , k} {w}_{ik} {y}_{ik} {‖ {\tilde{z}}_{ik} - {z}_{k} ‖}^{2}$$

wherein yᵢₖ represents the k^{th} cognitive function state label corresponding to the i^{th} sample, pᵢₖ is the probability of belonging to the k^{th} cognitive function state predicated by the model based on graph convolutional networks, z^{k} is a brain activation distribution priori graph under the current cognitive experimental paradigm, that is, a priori value of the activation degree of each brain region, \tilde{z}ᵢₖ is an activation degree value obtained by the model based on graph convolutional networks through learning, wᵢₖ is a mask containing the key brain region provided in the prior knowledge, the fitting degree of the model based on graph convolutional networks is calculated only in the mask, and α is a weight coefficient.

Further, in the step (8), the model based on graph convolutional networks is trained specifically as follows: randomly dividing the data set into a training set, a validation set and a test set by taking the subject as a unit, and taking the brain graph model obtained in the step (3) and the brain functional activity signals obtained in the step (4) as input of the model based on graph convolutional networks and taking the cognitive state at each time frame as a label to serve as output of the model based on graph convolutional networks, and training the model by using back propagation, where the training set is used for learning the model parameters; performing test on the validation set at the end of each training until the model converges or the preset training times are completed, finally saving a model with the best prediction effect on the validation set, and testing the generalization ability of the model on the test set; and extracting feature information of the last convolutional layer as graph representation information of the brain functional activity signals from the finally saved model based on graph convolutional networks, and generating a brain function activation graph of the subject in the corresponding cognitive state.

Compared with the background technology, the present disclosure has the following beneficial effects:

1. According to the present disclosure, the high-dimensional functional brain imaging data of the subject is mapped to the two-dimensional time-series matrix by the brain graph model, so on one hand, the feature dimension of the image data is greatly shortened, and the demand for computing resources and memory of the graphic card in the model training process is reduced; and on the other hand, a functional unit of the brain graph model is defined by the brain region with biological significance by using the mature brain atlas in existing researches in the dimension reducing process, for example, the brainnetome atlas based on the anatomical connection mode, the yeo atlas based on the functional organization mode and the Glasser atlas based on multi-modal imaging. The consistency of functional activity signals in the brain region is ensured maximally while dimension is reduced, and the problem of loss of brain functional activity information in the dimension reducing process is reduced. Compared with direct use of the downsampling technology on the space, the dimension reducing method can better save effective feature information for subsequent analysis, for example, distinguishing different cognitive functional states.

2. On the basis of the brain graph model, human brain hierarchical and modular organization mode is simulated by the graph convolutional neural network to realize a rapid and efficiency brain functional decoding algorithm: information interaction in the same brain network or functional submodule is considered, and information fusion between a plurality of functional networks can be finally realized by defining a high-order graph convolutional operator and using a multilayer graph convolutional neural network, so that the model is suitable for highly localized brain functions (such as finger movement), and has a good prediction effect on a high-grade cognitive function (such as working memory) involving multi-task integration.

3. Based on brain function decoding, the graph representation information of the brain functional activity generated by the model can be constrained by introducing the prior knowledge of the brain function activation mode while the prediction effect of the brain functional state is ensured, and the experienced brain functional activation mode can be reproduced, so that the graph representation information with biological significance is acquired. The information can be applied to the subsequent experimental task, for example, generating a brain functional activation graph of the subject and accurately predicting the brain functional region of the subject.

### BRIEF DESCRIPTION OF THE DRAWINGS

In order to more clearly illustrate the technical solutions of the present disclosure, the accompanying drawings needing to be used in the description of embodiments will be briefly described below. Obviously, the accompanying drawings described below are only specific embodiments recorded in the present application, and are not used to limit the protection scope of the present disclosure. For those of ordinary skill in the art, some other embodiments and accompanying drawings can be certainly obtained according to the following embodiments and accompanying drawings of the present disclosure without any creative effect.

FIG. 1 is a flow schematic diagram of a graph model-based brain functional alignment method;

FIG. 2 is a construction schematic diagram of a brain graph model; and

FIG. 3 is a structural schematic diagram of a model based on graph convolutional networks.

### DETAILED DESCRIPTION

To enable those skilled in the art to better understand the technical solution of the present application, the present disclosure is further described below with reference to the accompanying drawings. However, this is only some rather than all of the embodiments of the present application. Based on the embodiments of the present application, other embodiments obtained by other people in the art without any creative effort shall belong to the concept scope of the present disclosure.

The preferred embodiments of the present disclosure are described below with reference to the accompanying drawings.

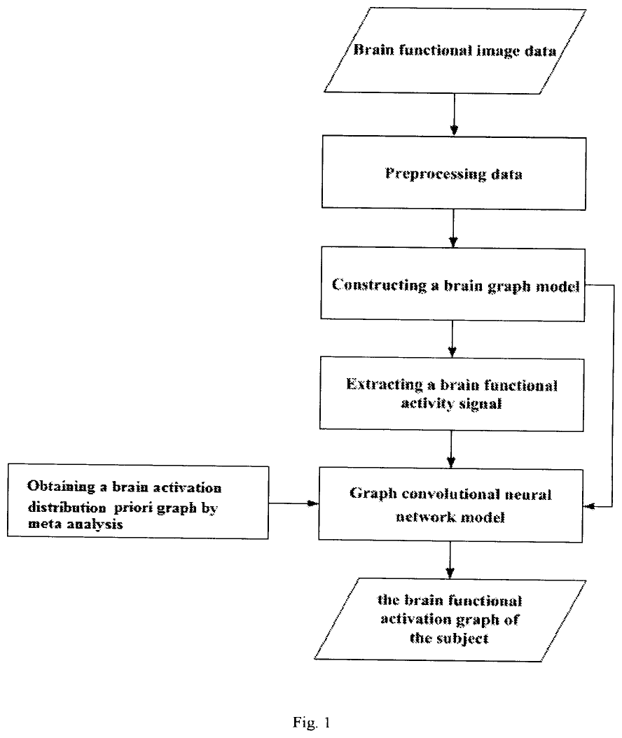

In general, the present disclosure provides a graph model-based brain functional alignment method. On the basis of completing the brain structural morphology registration, functional brain imaging data of all subjects in the same cognitive function state are mapped to the same representation space by using an artificial intelligent algorithm and supervised learning and under the guidance of distinguishing brain functional activity signals in different cognitive functional states, thereby ensuring that brain functional activation modes between different subjects have a good correspondence. The whole method flow is shown in FIG. 1, a brain graph model is constructed by taking functional brain imaging data of each subject in the specific cognitive function state as input, brain functional activity signals of each brain region are extracted on this basis to serve as a graph signal, and the graph signal is input into a model based on graph convolutional networks; meanwhile, priori knowledge of a brain functional activation mode corresponding to the cognitive function is obtained by a meta analysis method to generate a brain activation distribution priori graph, so that a brain functional activation mode of each subject is predicted while different cognitive function states are distinguished, the functional brain imaging data of each subject is mapped to a shared representation space suitable for a large-scale group, and accurate brain functional alignment between subjects is finally realized.

According to the method, by enhancing the functional correspondence between the subjects, the effect size of statistical test can be enhanced during group analysis, the number of subject samples required by scientific research can be reduced and the research cost can be saved; meanwhile, the graph representation information in the shared representation space can be used to accurately predict the brain function state and behavioral index of each subject.

The specific implementation process of the method includes the following steps:

(1) Acquiring task-based functional magnetic resonance imaging data from a Human Connectome Project using a computer to form a functional brain imaging data set. A task functional magnetic resonance imaging (task fMRI) data set is collected from a human connectome project (HCP, the connection address: https://db.humanconnectome.org/data/projects/HCP_1200), the set includes about 1200 healthy subjects, and various different cognitive experimental paradigms are completed. In this embodiment, the size of the used functional brain imaging data set and the distribution situation in each cognitive experimental paradigm are shown in the following table, wherein the total number of images refers to the total number of frames of three-dimensional functional brain imaging data and represents the amount of data used for brain functional state prediction based on a single frame (time window is 1 TR); the total number of cognitive experiments refers to the size of the data set used during brain functional state prediction by taking a single cognitive experiment as a unit; the duration of the cognitive experiment represents the duration of the shortest cognitive experiment in each cognitive experimental paradigm, which is calculated in seconds; and the category number of cognitive states is the target category number of brain functional state prediction in each kind of cognitive experimental paradigm. During implementation, an independent brain functional state prediction model is created for each kind of cognitive experimental paradigm. For example, for the working memory paradigm, the cognitive functional states (totally 8 different cognitive functional states, including four image recognition tasks such as face, scene, object and tool, and a combination of two memory tasks such as Oback and 2back) corresponding to the brain functional signal per 25 seconds are predicted through model training, the total data amount may reach to 17,360 samples, and the total data amount may reach 878,850 samples when a single frame of functional brain imaging data is selected for prediction.

| | | The | | Duration | The |
|---|---|---|---|---|---|
| | The | Total | The Total | of Each | Category |
| Cognitive | Number | Number | Number of | Cognitive | Number of |
| Experimental | of | of | Cognitive | Experiment | Cognitive |
| Paradigm | Subjects | Images | Experiments | (second) | States |
| Working | 1085 | 878,850 | 17,360 | 25 | 8 |
| memory | | | | | |
| Limb | 1083 | 615,144 | 21,660 | 12 | 5 |
| movement | | | | | |
| Language | 1051 | 664,232 | 16,816 | 10 | 2 |
| Social | 1051 | 575,948 | 10,510 | 23 | 2 |
| cognition | | | | | |
| Emotion | 1047 | 368,544 | 12,564 | 18 | 2 |
| processing | | | | | |
| Logical | 1043 | 483,952 | 12,516 | 16 | 2 |
| relation | | | | | |
| processing | | | | | |

(2) The functional brain imaging data of all subjects is registered to an image template of a common standard space based on structural morphology information, and the correspondence of the subjects on a brain anatomical structure is ensured. The specific operation is: firstly, the functional brain imaging data of each subject is registered to a space where a brain structure image of the subject is located through rigid body transformation or affine transformation to realize cross-modal registration in the same subject; secondly, the structure image of the subject is registered to a structure image template of a standard space by non-linear transformation, and a registration parameter of each subject to the standard space is saved; finally, the obtained registration parameter is applied to the functional brain image of the subject to realize registration from an subject space to the standard space and ensure the correspondence of all the subjects on the brain anatomical structure.

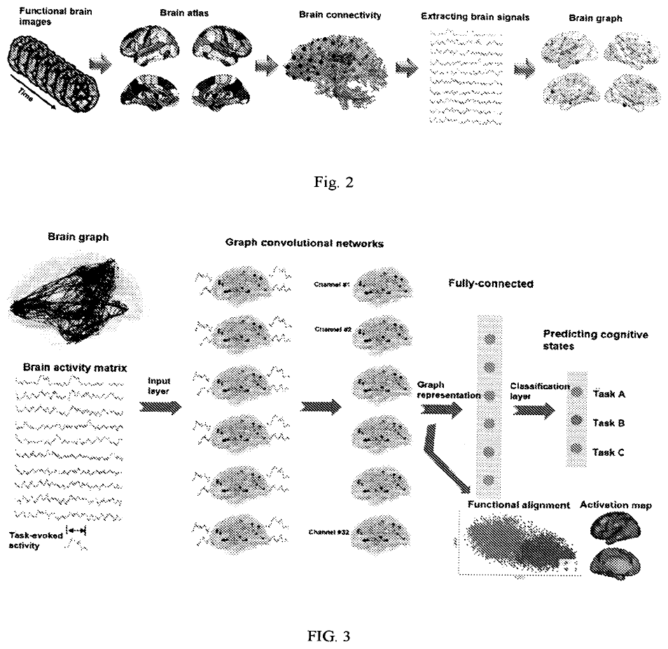

(3) A unified brain graph model under a standard space is created using brain atlas and brain connectomes. The specific construction process of the brain graph model is shown in FIG. 2: firstly, the whole cerebral cortex and subcutaneous substructure is divided into a plurality of spatially separated brain regions by using an existing brain atlas, including an anatomical, function and multi-modal brain atlas; secondly, the connection mode between different brain regions is calculated, including a brain anatomical connection based on diffusion magnetic resonance, a brain functional connection based on resting state functional magnetic resonance and a brain structural connection based on structural magnetic resonance and morphological feature covariation; finally, a brain graph model is constructed, wherein a node set V is formed by a brain region extracted by a brain atlas, and an edge set E is defined by brain connectome obtained through calculation; preferably, a node of the brain graph model is created by a brainnetome atlas, and the brain functional connection of the subject is calculated as an edge of the brain graph model.

(4) A brain functional signal is extracted; the original high-dimension functional brain image feature (such as four-dimensional functional magnetic resonance image data, the first three dimensions are spatial domain coordinates xyz, the fourth dimension is a time domain, representing the brain functional activity mode at different time points) is converted by the brain graph model obtained in the step (3) into a two-dimensional time-series matrix (the first dimension represents different brain regions and the second dimension represents different time frames), as shown in “brain functional signal extraction” in FIG. 2. Then, the derived time-series matrix as a graph signal is added to the brain graph model for representing the brain functional activity signals on each brain region. There are many different methods for calculating the graph signal, and the simple method includes: calculating the mean and variance, averaged time series and principal components of the brain functional activity signals in each brain region in the corresponding cognitive function state. Preferably, the mean time series serves as the representative brain functional activity signals in each brain region.

(5) A graph Laplacian matrix of the brain graph model is calculated L=I−D^{-1/2}AD^{-1/2}, wherein I represents a unit matrix, A represents a connection relationship between nodes, may be a binary adjacency matrix, or may be a weighted brain connection strength or connection probability, and D represents the connectivity on each node. Spectral decomposition of the graph Laplacian matrix Lv=λv is used to obtain a eigenvalue {λᵢ} and a feature vector {vᵢ}, and graph Fourier transformation \tilde{x}=U^{T}x and inverse transformation may be performed x=U\tilde{x} on this basis, wherein the eigenvalue {λᵢ} represents different frequency bands of graph Fourier transformation, the corresponding feature vector {vᵢ} represents a transformation base U=(v₁, . . . , vₙ) under a corresponding frequency band, n represents the total number of brain regions, represents the original graph signal, \tilde{x} represents the graph signal after transformation, and T represents transpose operation; and the graph signal may be transformed from an image space domain where the functional brain imaging data is located to a spectral domain defined by the brain graph model through graph Fourier transformation, and graph convolution operator operation is continuously performed in the spectral domain x*_{G}g_{θ}=Ug_{θ}U^{T}x, represents g_{θ} a convolution kernel, and *_{G} represents convolutional operations defined on the brain graph model, and constructing a model based on graph convolutional networks by taking the graph convolution operations.

(6) For the researched cognitive experimental paradigm, a meta analysis method is used to obtain priori knowledge of a brain activation mode of the cognitive function from the published related research, and a brain activation distribution priori graph is generated. The specific steps are as follows: the coordinates of a center point (peak point) of the brain region that is significantly activated in the researched cognitive experimental paradigm are extracted from the existing research by using the common meta analysis software such as brainmap database (brainmap.org), a smoother brain activation distribution map (ALE map) is generated on each center point by virtue of Gaussian kernel, and finally, the final brain activation distribution prior graph is generated by a statistical test method. For example, for the working memory paradigm, a total of 309 published related researches are obtained by searching the brainmap database, including 6912 center point coordinates of 4728 subjects. The brain activation distribution prior graph is generated by the ALE algorithm, and the mask of the significantly activated brain region of the working memory paradigm is obtained by setting the threshold of the significant activation degree, such as z≥3.0, for subsequent analysis.

(7) An additional loss function (mean square error loss function) is added to a target function (cross entropy loss function) in the original model based on graph convolutional networks for predicting the brain functional state by combining the prior knowledge in the step (6), wherein the added loss function is used to represent the correspondence degree between the activation degree of each brain region and the brain activation distribution priori graph; and the graph representation information is constrained to fit the prior knowledge activated by the brain function on the key brain region as much as possible while the brain functional state is predicted; and under this frame, the target function Loss of the model based on graph convolutional networks with brain functional activation prior constraint is:

$$Loss = {\sum }_{i , k} {y}_{ik} log ( {p}_{ik} ) + α {\sum }_{i , k} {w}_{ik} {y}_{ik} {‖ {\tilde{z}}_{ik} - {z}_{k} ‖}^{2}$$

wherein yᵢₖ represents the k^{th} cognitive function state label corresponding to the i^{th} sample, pᵢₖ is the probability of belonging to the k^{th} cognitive function state predicated by the model based on graph convolutional networks, zₖ is the group prior value of a brain activation distribution in each brain region under the current cognitive experimental paradigm, \tilde{z}ᵢₖ is brain activation degree values obtained by the model based on graph convolutional networks through learning, wᵢₖ is a brain mask containing the significantly activated brain regions provided in the group priors, the fitting degree of the model based on graph convolutional networks is calculated only in the mask, and α is a weight coefficient, and the empirical value is 0.001.

(8) The model based on graph convolutional networks is trained, as shown in FIG. 3, brain functional activity signals of different subjects are mapped to the same shared representation space to realize the brain functional alignment between the subjects, and the brain functional activation mode of the subject is predicted; the data set is randomly divided into a training set (70%), a validation set (10%) and a test set (20%) by taking the subject as a unit; and model training is performed by taking the brain graph model obtained in the step (3) and the brain functional activity signals obtained in the step (4) as input of the model based on graph convolutional networks and taking the cognitive state at each time frame as a label to serve as output of the model based on graph convolutional networks through backpropagation. The training set is used to learn the model parameters, test is performed on the validation set at the end of the training until the model converges or the preset training times are completed (for example, the number of the preset training times is 200), and finally, the model with the best prediction effect (the prediction accuracy rate is the highest and is consistent with the prior knowledge) on the validation set is saved, and the generalization ability of the model is tested on the test set. Feature information of the last convolutional layer is extracted from the finally saved model based on graph convolutional networks to serve as graph representation information of the brain functional activity signals, so that mapping from the high-dimensional functional brain imaging data space to the low-dimensional representation space is realized, the brain functional activation mode of the subject can be represented, the brain functional activation graph of the subject in the corresponding cognitive state is generated, and the brain function activation graph of the subjects is displayed on a displayer.

The above is only the preferred embodiment of the present application. The present application is not limited to the specific embodiments described herein, and can cover the widest scope consistent with the principles and novel features disclosed herein.

What is claimed:

1. A graph model-based brain functional alignment method, comprising:
(1) acquiring task-based functional magnetic resonance imaging data from a Human Connectome Project using a computer to form a functional brain imaging data set, and recording a cognitive function state on each time frame in the functional brain imaging data set according to the design of a cognitive experimental paradigm;
(2) registering functional brain imaging data of all subjects to an image template of a common standard space based on structural morphology information, and ensuring the correspondence a brain anatomical structure across all subjects;
(3) creating a unified brain graph model in the standard space using brain atlas and brain connectomes;
(4) converting an original feature of high-dimensional brain function images into a two-dimensional time-series matrix by using the brain graph model in the step (3), wherein the first dimension represents different brain regions, and the second dimension represents different time frames; adding the derived time-series matrix as a graph signal into the brain graph model for representing brain functional activity signals on each brain region;
(5) calculating a graph Laplacian matrix of the brain graph model, obtaining eigenvalues and eigenvectors of the graph Laplacian matrix using spectral decomposition, transforming the graph signal in the step (4) from the original image space domain to a spectral domain defined by the brain graph model through graph Fourier transformation, applying graph convolution operation and spectral analysis on the brain functional activity signal, and learning graph representations of brain activity by creating a model based on graph convolutional networks;
(6) using a meta analysis method to obtain priori knowledge under a brain functional activation paradigm, and generating a brain activation distribution priori graph;
(7) adding a term in the loss function for representing the correspondence degree of the brain activation distribution priori graph in each brain region in a target function of the model based on graph convolutional networks by combining the prior knowledge in the step (6);
(8) training the model based on graph convolutional networks, extracting feature information of the last convolutional layer as graph representation information of the brain functional activity signals, wherein the graph representation information maps functional brain imaging data of different subjects to the same representation space to realize brain function alignment of the subjects and generate a brain function activation graph of the subjects; and
displaying the brain function activation graph of the subjects on a displayer.

2. The graph model-based brain functional alignment method according to claim 1, wherein in the step (2), the specific operation of registration based on the structural morphology information is: cross modal registering the functional brain imaging data of each subject to individual structural image space; registering non-linear transformation of the structure images of the subject to a structure image template of a standard space, and saving a registration parameter of each subject to the standard space; and applying the registration parameter to the functional brain imaging data of the subject to realize the registration from a subject space to the standard space.

3. The graph model-based brain functional alignment method according to claim 1, wherein in the step (3), the specific creating process of the brain graph model is: parcellating the whole cerebral cortex and subcutaneous substructure into spatially separated brain regions by using a well-established brain atlas; calculating the connectivity pattern between different brain regions using dMRI or fMRI; and constructing a brain graph model, wherein a node set V is formed by brain regions extracted by the brain atlas, and an edge set E is defined by brain connectome obtained through calculation.

4. The graph model-based brain functional alignment method according to claim 3, wherein the brain atlas comprises an anatomical, function and multi-modal brain atlas; and the brain connectome comprises an anatomical connectivity derived from diffusion tractography using diffusion MRI, a functional connectivity based on resting-state functional MRI, and a structural connectivity based on structural covariance using structure MRI and morphological feature covariance.

5. The graph model-based brain functional alignment method according to claim 1, wherein the graph signal is calculated as follows: calculating the mean value and variance, averaged time series and principal components of the brain functional activity signals in each brain region in the corresponding cognitive function state.

6. The graph model-based brain functional alignment method according to claim 1, wherein in the step (5), the spectral analysis of the brain functional activity signals is specifically as follows:
calculating the graph Laplacian matrix of the brain graph model L=I−D^{-1/2}AD^{-1/2}, wherein I represents a unit matrix, A represents a connection relationship between nodes, and D represents the connectivity on each node; obtaining the eigenvalues {λᵢ} and eigenvectors {vᵢ} by using spectral decomposition of the graph Laplacian matrix Lv=λv, and performing graph Fourier transformation \tilde{x}=U^{T}x on the basis U=(v₁, v₂, . . . vₙ), wherein the eigenvalue {λᵢ} represents different frequency bands of graph Fourier transformation, the eigenvector {vᵢ} represents the transformation under the corresponding frequency band, n represents the total number of nodes, x represents the original graph signal, \tilde{x} represents the transformed signal, and T represents the transpose operation; and transforming the graph signal from an image space to a spectral domain defined by the brain graph model through graph Fourier transformation, and applying graph convolution operator operation in the spectral domain x*_{G}g_{θ}=Ug_{θ}U^{T}x, wherein gθ represents a graph convolutional kernel, and *_{G} represents the convolutional operation defined on the brain graph model, and constructing the model based on graph convolutional networks based on the graph convolution operations.

7. The graph model-based brain functional alignment method according to claim 1, wherein in the step (6), the calculation of the group priors of the brain activation comprises: using meta analysis tools to extract the peak coordinates of significantly activated brain regions under the cognitive task or using the same experimental paradigm from an existing database, generating a Gaussian smoothed brain activation map on each peak, and generating a prior distribution of brain activation by using a statistical analysis.

8. The graph model-based brain functional alignment method according to claim 1, wherein in the step (7), a target function of the model based on graph convolutional networks comprises two items: the first item is a cross entropy loss for predicting the cognitive state at each time frame, and the second item is a masked mean square error loss function for constraining graph representation information to fit priori knowledge activated by a brain function on a key brain region as much as possible.

9. The graph model-based brain functional alignment method according to claim 8, wherein the target function Loss of the model based on graph convolutional networks is specifically as follows:
$$Loss = {\sum }_{i , k} {y}_{ik} log ( {p}_{ik} ) + α {\sum }_{i , k} {w}_{ik} {y}_{ik} {‖ {\tilde{z}}_{ik} - {z}_{k} ‖}^{2}$$
wherein yᵢₖ represents the k^{th} cognitive function state label corresponding to the i^{th} sample, pᵢₖ is the probability of belonging to the k^{th} cognitive function state predicated by the model based on graph convolutional networks, zₖ is the group prior value of a brain activation distribution in each brain region under the current cognitive experimental paradigm, \tilde{z}ᵢₖ is brain activation degree values obtained by the model based on graph convolutional networks through learning, wᵢₖ is a brain mask containing the significantly activated brain regions provided in the group priors, the fitting degree of the model based on graph convolutional networks is calculated only in the mask, and α is a weight coefficient.

10. The graph model-based brain functional alignment method according to claim 1, wherein in the step (8), the model based on graph convolutional networks is trained specifically as follows: randomly dividing the data set into a training set, a validation set and a test set by taking the subject as a unit, and taking the brain graph model obtained in the step (3) and the brain functional activity signals obtained in the step (4) as input, taking the cognitive state at each time frame as a label to serve as output of the model based on graph convolutional networks, and training the model by using back propagation, wherein the training set is used for learning the model parameters; performing test on the validation set at the end of each training until the model converges or the preset training times are completed, and finally saving a model with the best prediction effect on the validation set, and testing the generalization ability of the model on the test set; and extracting feature information of the last convolutional layer as graph representation information of the brain functional activity signals from the finally saved model based on graph convolutional networks, and generating a brain function activation graph of the subject in the corresponding cognitive state.
